(19) United States
(12) Patent Application Publication
(10) Pub. No.: US 2007/0148754 A1
(43) Pub. Date: Jun. 28, 2007

(54) Methods and compositions for improving plant growth

(76) Inventors: John Charles Marrelli, La Quinta, CA (US); Don A. Phillips, Caspar, CA (US)

Correspondence Address: SCULLY SCOTT MURPHY & PRESSER, PC, 400 GARDEN CITY PLAZA, SUITE 300, GARDEN CITY, NY, 11530, US

(21) Appl. No.: 11/633,362

(22) Filed: Dec. 4, 2006

Related U.S. Application Data
(60) Provisional application No. 60/742,107, filed on Dec. 2, 2005.
(60) Provisional application No. 60/749,392, filed on Dec. 12, 2005.

(51) Int. Cl. C12N 1/20

(52) U.S. Cl. 435/252.3; 435/253.6

(57) ABSTRACT

The present invention has surprisingly discovered that certain spore-forming microorganisms, present in soil, plants and other forms of organic matter, survive excessive heat of a fire within soil or carbonized wood. It has been determined that such microorganisms stimulate plant growth, enhance the nutritional value of plant products, and incorporate carbon dioxide. Accordingly, the present invention provides methods for isolating and identifying plant growth-stimulating microorganisms from soil and from carbonized organic materials. The present invention also provides compositions and methods useful for enhancing plant growth and nutritional properties and for producing DNA-enhanced plants which may be consumed by human individuals for enhancing human DNA. The compositions and methods of the present invention are also useful for improving soil. The invention also provides methods for using charcoal as a carrier to promote plant growth, and to transfer and relocate desirable microorganisms from one ecosystem to another.

### CROSS-REFERENCE TO RELATED APPLICATIONS

This application claims priority from U.S. Provisional Application No. 60/742,107, filed on Dec. 2, 2005 and U.S. Provisional Application No. 60/749,392, filed on Dec. 12, 2005.

### FIELD OF THE INVENTION

The present invention relates to identification and isolation of plant growth-stimulating microorganisms. In particular, the invention relates to isolation of fire-climax microorganisms from materials that encapsulate and protect such microorganisms from excessive heat, such as carbonized plant materials, oceanic waste, and soil. The present invention also relates to the use of such microorganisms for enhancing plant growth and nutritional properties through remediation and amendment of soil. The invention further relates to the use of charcoal to promote growth of microorganisms and to transfer and relocate desirable microorganisms from one ecosystem to another.

### BACKGROUND OF THE INVENTION

Current conventional farming methods used to improve crop yields on marginal farming lands generally exploit the excessive use of detrimental fertilizers and pesticides. Chemical fertilizers, in particular, are applied in increasing amounts to provide a source of nitrogen, phosphorous and potassium, as well as other minerals and micronutrients, in forms that are accessible to plants. Chemical fertilizers have acidified the soil, and deposited high levels of heavy metals and salts. Synthetic urea and other chemicals cause further imbalance by rendering essential nutrients and minerals inaccessible to plants. Overuse of fertilizers and pesticides results in an imbalance of essential nutrients in the amended soils, eventually rendering the land unsuitable for farming, and at the extreme, the inability to maintain plant life. Irrigation and rainwater leach applied fertilizers and pesticides into waterways, causing eutrophication of lakes, rivers and other local water sources, contributing substantially to water pollution and creating non-drinkable or toxic water sources.

Organic farming methods have been introduced over the past few decades in an effort to reduce the negative impact of conventional fertilizers and pesticides on the environment. Organic farming practices, however, have introduced other problems. For example, fertilizers used in organic farming can contain heavy metals, organic pollutants, and microbial pathogens. Rotenone, a pesticide made from natural products and used in organic farming, is capable of killing dopamine-producing neurons, resulting in motor deficits. Rotenone has been shown to produce parkinsonian symptoms in rats. (Renner, R. “From Flush to Farm,” Scientific American, 10/2002; Karow, J., “Pesticides and Parkinson's,” Scientific American, Nov. 6, 2000; Lozano, A. M. and Kalia, S. K., “New Movement in Parkinson's: Environmental Culprits,” Scientific American, p. 71, Jun. 27, 2005.)

There is a need, therefore, for methods and compositions that resolve current imbalances in soil nutrients and reduce levels of toxic chemicals, as well as create self-sustaining farming lands that impact minimally on the surrounding environment. “Terra preta” soils are rich soils prized for their enhanced fertility and productivity. Found in pockets of the Amazon rainforest, they contain high levels of organic matter and carbon. Terra preta soils have also been observed to contain higher levels of nutrients, including levels of nitrogen, phosphorous, calcium and potassium, and to have greater nutrient and moisture-holding capacity than non-terra preta soils. Sombroek, W G et al., Ambio, 22:417-426 (1966); Smith, N. J. H., Ann. Assoc. Am. Geogr. 70:553-566 (1980); Zech, W., et al., In: McCarthy, P. et al., eds., American Society of Agronomy and Soil Science Society of America, Madison, Wis., pp. 187-202 (1990). Like terra preta soils, terra mulata soils display a dark grayish brown color and contain elevated levels of soil organic matter.

Despite the intense research surrounding the formation of terra preta soils, the identity and functions of bacteria and fungi in terra pretas and terra mulata soils remain unknown. See, e.g., Lehmann, J. et al., eds., “Amazonian Dark Earths”, Kluwer Academic Publ. (2003). For example, although scientists have recognized the prevalence of charcoal as a key ingredient in terra preta soils, and have known bacteria and other microorganisms to exist in the soil, the identity and functions of bacteria and fungi in terra pretas and terra mulata soils are unknown. Glaser, B. et al., Biol. Fertil. Soils 35:219-230 (2002); Lehmann et al. (2003).

### SUMMARY OF THE INVENTION

The present invention is based on the surprising discovery that certain spore-forming fire-climax microorganisms, or “fire-climax microbes” as referenced herein, present in organic materials such as soil and plants, survive extreme conditions such as excessive heat of a fire. The plants die in a fire, but these fire-climax microbes form spores and survive within an encapsulating material, such as soil and carbonized plant materials by virtue of the insulating properties of carbonized plant materials and soil, and repopulate soil and plants once conditions become favorable.

Accordingly, the present invention provides a reproducible method for isolating and selecting such fire-climax microbes, as well as microbes therefore isolated. It has also been determined that these fire-climax microbes stimulate plant growth, enhance the nutritional value of plant products, and incorporate carbon dioxide. The present invention provides the unique recognition that carbonized wood serves as a repository of plant-growth-stimulating fire-climax microbes, and therefore can be employed as a carrier to relocate plant-growth-stimulating fire-climax microbes for ecosystem reconstruction. This invention also demonstrates that charcoal promotes vegetative growth of fire-climax microbes, possibly by protecting the fire-climax microbes through the adsorption of deleterious chemical factors, which are produced during the sporulation process. It is further proposed in accordance with the present invention that the remarkable fertility of terra preta soils is attributable to natural and man-made fires in Amazon rainforest, which generated extensive carbonized wood and permitted fire-climax microbes to survive and flourish. Based on the new understanding of the relationship between charcoal and fire-climax microbes, the present invention provides methodologies for employing a natural reset button to correct environmental damage created by agriculture and to initiate the type of molecular self-assembly that occurs in natural systems following a fire.

In one embodiment, the present invention provides a method for isolating fire-climax microbes from a carbonized organic material, particularly a carbonized plant material, e.g., charcoal, by inoculating a growth medium with the carbonized material containing spores of the fire-climax microbes, and maintaining the growth medium at an appropriate temperature for a period of time sufficient to permit vegetative growth of the fire-climax microbes within the medium. A mixture of multiple strains as well as single, isolated strains of fire-climax microbes can be obtained.

The present invention also provides a process for producing carbonized materials, particularly carbonized plant materials, which process mimics the conditions in a natural fire and permits a reproducible isolation and selection of fire-climax microbes. In a specific embodiment, the process involves heating or burning an organic material under conditions such that carbonization progresses to the extent just prior to extinction of fire stable bacteria. For example, the heating can be conducted at a temperature and for a period of time that are about 1 BTU away from the temperature and time that would have resulted in extinction of fire stable bacteria. In a preferred embodiment, the process involves heating or burning a plant material at a temperature of at least 200° C., or preferably at least 400° C., or more preferably at about 600° C., for a period of time of from about 5 to about 20 minutes, preferably about 10 to about 15 minutes, to produce a carbonized plant material.

In another embodiment, the present invention provides a method for isolating fire-climax microbes from soil by boiling a liquid suspension of a soil containing spores of fire-climax microbes, inoculating a growth medium with an aliquot of the boiled suspension, and maintaining the growth medium at an appropriate temperature for a period of time sufficient to permit vegetative growth of fire-climax microbes within the medium.

In a further embodiment, the present invention provides a method for identifying a fire-climax microbe strain that stimulates plant growth. Individual strains of fire-climax microbes are isolated from soil or charcoal and are screened for the ability to stimulate plant growth.

In still another embodiment, the present invention provides isolated fire-climax microbes that produce spores which survive at a temperature of at least 200° C. or even up to about 600° C. or higher. Preferred fire-climax microbes include isolates of Brevibacillus centrosporus, particularly, HAB7, and isolates of Bacillus megaterium, such as AC9.

In one embodiment, the present invention provides compositions containing fire-climax microbes that produce spores which survive at a temperature of at least 200° C. or even about 600° C. The compositions of the present invention are useful for enhancing plant growth and improving soil quality, and can be manufactured as fertilizer compositions.

In another embodiment, the present invention provides a method for enhancing plant growth by growing cultivars of a plant in a plant cultivation medium supplemented with at least one fire-climax microbe of the present invention.

In still another embodiment, the present invention provides a method for producing nutritionally-enhanced plant products by growing cultivars of a plant in a plant cultivation medium supplemented with at least one fire-climax microbe of the present invention.

In a further embodiment, the present invention provides methods of enhancing growth and/or nutritional value of a plant by growing cultivars of a plant in a plant cultivation medium supplemented with charcoal. Preferably, the charcoal to be employed in the methods has been selected for containing desired fire-climax microbes.

The plants and plant products produced based on the methods of the present invention form another embodiment of the present invention. Plant products produced according to the present invention are beneficial to animals that ingest them, because the fire-climax microbes increase the content of phytochemicals in the plant (Examples X and XI) and are believed to stimulate the animals' immune system.

In a further embodiment, the present invention provides a method for improving the plant growth-stimulating property of a soil and for increasing biodiversity in a soil by employing the fire-climax microbes or compositions containing the fire-climax microbes identified in accordance with the present invention.

Moreover, the present invention provides methods for enhancing solar energy conversion by plants, and methods for reducing pollution by enhancing the efficiency of mineral nutrient utilization by plant.

The fire-climax microbes of the present invention and/or charcoal containing fire-climax microbes can also be introduced to non-agricultural lands, including aesthetic wild lands, urban green belts and golf courses.

### BRIEF DESCRIPTION OF THE DRAWINGS

FIG. 1. Comparison of Partial 16S rDNA Sequences from Microbes Isolated from Charcoal to Known Bacterial Sequences. This figure shows the closest bacterial sequence matches and the corresponding percent sequence differences. Four isolates, CP1-2, CP1-6, CP1-13, and CP1-14 were not identifiable. The “closest match” designates for each of these isolates differed in sequence by >5%, thus were concluded unlikely to be the same. (See Example I)

FIG. 2. Comparison of Partial 16S rDNA Sequences from Microbes Isolated from Charcoal to GenBank Sequences. This figure shows the closest GenBank sequence matches found for the four unmatched isolates. (See Example I)

FIG. 3A-3B. Analysis of 16S rDNA Sequences from Microbes Isolated from Almond Charcoal. One of the 29 isolates analyzed (see 3B) was identified as Brevibacillus centrosporus. (See Example II)

FIG. 4. Analysis of Partial 16S rDNA Sequences from Microbes Isolated from Soil. This figure shows, for each of samples DAP-1 to DAP-6, the closest bacterial sequence match and the corresponding percent difference in sequence between the sample strain and the closest match based on 500 base pair 16S rDNA sequencing. DAP-2 was determined be Brevibacillus centrosporus. (See Example III)

FIG. 5. Complete 16S-rDNA sequence obtained for HAB7. (See Example III)

FIG. 6. Effect of Charcoal on Bacterial Growth. The graph shows that the charcoal had enhanced bacterial growth approximately 20-fold on Day 3 and 8-fold on Day 5 as compared with untreated control cultures. The Day 5 effect, determined by measuring viable cells in each of three replicate cultures for each treatment, was highly significant (P≦0.01). The Day 3 effect was measured with a single replicate in each treatment. (See Example V)

FIG. 7. Carbon Fixation by HAB7. HAB7 cells incorporated CO₂ to a level of approximately 0.25% of total carbon (black circles). Increases in ¹³C content between 12 and 18 hours and between 12 and 36 hours were highly significant (p≦0.001). Increased CO₂ concentration (black triangles) promoted cell growth (mass) by 50% at T₁₂ (12 hour) (P≦0.01) and by 12% at T₃₆ (36 hour) (P≦0.01) relative to ambient atmospheric CO₂ (open triangles). (See Example VI)

FIG. 8. Effect of Urea on Growth of HAB7. The growth of HAB7 cultured in the presence or absence of urea for thirty hours was evaluated. The figure shows that the urea significantly inhibited HAB7 growth. (See Example X)

### DETAILED DESCRIPTION OF THE INVENTION

For clarity of disclosure, and not by way of limitation, the detailed description of the invention is divided into the following subsections which describe and illustrate certain features, embodiments or applications of the invention.

Characteristics of Fire-Climax Microbes of The Present Invention

The term “fire-climax microbes”, as used herein, refers to highly evolved, spore-forming bacteria that associate beneficially with plants and have a special relationship with fire. Fire-climax microbes have the ability to adapt to environmental changes, and produce spores that withstand temperatures higher than those normally associated with life-sustaining conditions. All fire-climax microbes, for example, survive in soil at 200° C., 400° C., or even 600° C. as fire passes over them, and many fire-climax microbes can be found in pyrrolized carbon substrates, such as charcoal or other forms of carbonized plant materials, as well as other forms of carbonized organic materials, produced by heating at much higher temperatures (e.g., 500° C. or even 600° C.).

According to the present invention, fire-climax microbes derive four benefits from fire. First, fire weakens spore walls, which then can take up water needed to trigger spore germination. Second, fire produces charcoal, which maintains fire-climax microbes in a state of persistent vegetative growth by adsorbing chemical factors favoring sporulation (Gonzalez-Pastor et al., Science 301: 510-513, 2003) and thus promoting immediate bacterial growth. Third, fire kills many microorganisms that compete with fire-climax microbes. Finally, fire creates a fertile platform for fire-climax microbes by releasing mineral nutrients from plant materials. The fire-climax microbial community flourishes after a fire by colonizing new plant roots. Eventually, as the products of fire—charcoal, high nutrient levels, and reduced competition—disappear, fire-climax microbes form spores and rest until the next cycle of fire and regrowth occur. In this sense, fire-climax microbes represent a microbial version of the commonly recognized fire climax plant species, which also awaken and grow profusely after a fire.

According to the present invention, fire-climax microbes exist within plants and benefit the host plants by stimulating the production of phytochemicals important for plant growth and protection. Additionally, fire-climax microbes stimulate key metabolic pathways in plants, including root exudation and respiration, which help direct carbon flow from aboveground plant organs to roots. This stimulation of root growth in turn increases the availability of mineral nutrients and water. Further, various fire-climax microbes stimulate plant growth by reducing N₂ to ammonia, inhibiting growth of plant pathogens, solubilizing phosphate, and incorporating carbon dioxide. Without intending to be bound by any particular theory, it is believed that the stimulatory effects of fire-climax microbes on plants are achieved epigenetically. Fire-climax microbes may up-regulate expression of selected genes in host plants, resulting in “gene-enhanced” or “DNA-enhanced” plants, characterized by, e.g., enhanced growth and production of phytochemicals. See reviews of epigenetics by Grant-Downton and Dickinson, Annals Botany 96: 1143-1164 (2005), Part 1; and Annals Botany (October, 2005), Part 2.

Further according to the present invention, fire-climax microbes also exist in virgin soil. Survival of fire-climax microbes as spores during a fire and their subsequent growth in the presence of moisture place the fire-climax microbes at the head of the line for colonizing new plant roots.

Isolation and Identification of Fire-Climax Microbes

In one embodiment, the present invention provides methods for isolating fire-climax microbes from carbonized organic matters.

Organic matters are materials of biological origin which contain (1) fire-climax microbes, (2) their DNA, and (3) inert substrates consisting of carbon and other elements associated with living organisms, such as oxygen, hydrogen and nitrogen. Examples of organic matters suitable for isolating fire climax microbes include plant materials, oceanic waste materials such as fish meals and algae and organic matters derived from soil.

Carbonization refers to the conversion of an organic substance into a residue containing primarily carbon. Generally such residues are composed of mixtures of polycyclic, aromatic carbon molecules.

In a specific embodiment, the carbonized material is charcoal, which can be obtained from nature or can be artificially produced by a number of methods known to those of skill in the art. For example, charcoal can be produced from dry wood in a modem, low-emission wood stove that has an effective damper system. One stove suitable for this purpose has an interior dimension of 20″×20″×15″ (W×D×H). The operator builds a fire using kindling, such as split redwood, and several split logs of pine or oak. The pine or oak logs should be replenished several times and burned with the damper completely open to heat the stove adequately and to generate a suitable bed of coals. After a period of approximately 3 to 4 hours, a bed of glowing coals approximately 5″ deep should be present on the bottom of the stove.

Wood logs for producing charcoal can be either intact or split, but experience teaches that intact logs give a higher yield of charcoal. Three or four logs chosen for charcoal production should be placed on the live (glowing orange) coals with the damper completely open. The new logs typically will burst into flame within about 10 minutes, depending on moisture content of the wood, and an intense fire should result within about 20 minutes. Several minutes after all logs are enveloped in flames, the damper should be closed completely to reduce oxygen availability. The fire will quickly subside, and the logs will char and burn simultaneously. A stove containing logs under the conditions described here can be allowed to remain overnight without further supervision. In the morning, the stove will be cool, and charred remnants of the logs will be present. A charred log can be broken into pieces of smaller charcoal immediately.

Yields of charcoal on a weight basis are highly variable. An experienced collier can obtain a 25% yield, but a more typical yield under the conditions described here is about 15-20%.

To release fire-climax microbes within charcoal, charcoal fines, dust-like particles of approximately 2 mm-15 mm in diameter, are separated from the larger pieces of charcoal. The fine charcoal particles are then used to inoculate a growth medium selected from any media suitable for bacterial cultures. Inoculation can be achieved by adding charcoal particles directly into a liquid growth medium. Alternatively, solid growth medium can be used, in which case, charcoal particles can be added to the growth medium during the preparation of the medium and prior to its solidification (for example, shortly after the medium is autoclaved). The growth medium inoculated with charcoal particles is then maintained at an appropriate temperature in the range of 5° C.-55° C. for a period of time sufficient to permit vegetative growth of fire-climax microbes within the medium. Fire-climax microbes so produced can be easily separated from the growth medium for further use.

Individual strains of fire-climax microbes can also be isolated, if desired. For example, charcoal particles can be added to a liquid suspension of a growth medium before the medium solidifies. To obtain single colonies on the plate, charcoal is added to the liquid suspension at a concentration of about 0.5% to about 10%, preferably about 1% to about 5%, and more preferably about 3% (w/v). The medium suspension is then poured into Petri plates to solidify. Single colonies representing individual fire-climax microbe strains will develop on the plates and can be collected for further use or analysis. 16S rDNA of the colonies can be sequenced to identify the fire-climax microbes at the species level.

In another embodiment, the carbonized material for isolating fire climax microbes is produced by heating an organic material containing fire climax microbes or spores thereof, such as shredded bark tissues and wood of a plant, or oceanic organic waste, under conditions such that carbonization progresses to the extent just prior to extinction of fire stable bacteria. For example, the heating can be conducted under conditions about 1 BTU away from the conditions that would have resulted in extinction of fire stable bacteria.

In a specific embodiment, an organic material, such as shredded bark tissues and wood of a plant, or oceanic organic waste, is heated at an extremely high temperature (i.e. heat treatment), e.g., at about 600° C., for a time period, preferably about 10-15 minutes, and most preferably at 600° C. for 10 minutes. To achieve consistent and complete carbonization, the heating process can be performed using a furnace having a small chamber, as exemplified in Example III below. Wood in Example III could have burned away to nothing, but the carbonization process was stopped at about 10 minutes to recover bacterial spores just before they became extinct, i.e., no viable spores remained in the next sample (heated at 600° C. for 15 minutes) collected.

By “about 600° C.”, it is meant a temperature in the range of 585° C. to 615° C., preferably 590° C. to 610° C., or more preferably 595° C. to 615° C. The precise temperature needed to achieve the desired degree of carbonization (i.e., just prior to extinction of the fire stable microbes) may depend on the specific density, moisture content, number or amount, uniformity, shape, size, consistency, chemical composition, oxygen level, pressure, chemical treatment (if any), of the organic material that encapsulates the fire stable microbes.

The thermal conditions, including the temperature and the time period, should be such to yield residues weighing approximately 10-20% of the starting material. In connection with the present invention, the thermal conditions may be appropriately defined by the energy required to release, recover, isolate or obtain fire climax microbes from organic matters. The formula, time multiplied by temperature, is proportional to the energy required to release, recover, isolate or obtain fire-climax microbes from organic matters. In the case where heat treatment of organic matters for 10 minutes at 600° C. supplies 100% of the energy required to release, recover, isolate or obtain fire climax microbes, heat treatment of organic matters for 5 minutes or 15 minutes, at the same temperature, will provide 50% and 150% of the energy value, respectively. Therefore, according to the present invention, the thermal conditions suitable to release, recover, isolate or obtain fire climax microbes from organic matters are those that supply, for example, 51% to 149% of the energy.

The resulting carbonized material is then used to inoculate a growth medium suitable for bacterial cultures, as discussed above in connection with charcoal.

In another embodiment, the present invention provides methods for isolating fire-climax microbes from soil. In accordance with this embodiment, soil can be suspended in water, for example, at a ratio of 1:1 (v/v). The suspension is then placed in a boiling water bath for about 2-4 minutes, preferably about 3 minutes. An aliquot is taken from the suspension and plated on a solid growth medium suitable for bacterial growth. An example of such growth medium is TY medium (Bacto Tryptone (5 g/1), Bacto Yeast Extract (3 g/l) and CaCl₂ (1.3 g/l), with Bacto Agar (15 g/l)). Individual bacterial colonies will develop on the solid growth medium, and can be further subcultured if necessary to obtain single bacterial strains.

Bacteria isolated as described above can be further screened for the ability to enhance plant growth and productivity. Plant growth and productivity can be determined based on shoot dry weight, seed yield, growth of root, and/or fruit yield or size. Plants that can be used as a test medium include virtually any plant grown in soil. Exemplary plants include commodity grain crops (e.g. corn, wheat, and soybeans), and sorghum. Other examples include raw agricultural commodity crops, including fruiting and nut-bearing trees (e.g., almonds), soybeans, peanuts, grapes, apples, berries (strawberries, blackberries, raspberries), tubers (e.g. potatoes, sweet potatoes), corn, cereal grains (e.g. wheat, rice, rye), tomatoes, onions, cucurbits (e.g., watermelon, cucumber, and cantaloupe), leafy vegetables (e.g., lettuce, spinach, endive), cotton and other commodity crops.

Isolated Strains of Fire-Climax Microbes

In another embodiment, the present invention provides fire-climax microbes isolated based on the methodology of the present invention described above.

Fire-climax microbes isolated according to the methodology of the present invention are bacteria that produce spores that withstand high temperatures of at least 200° C., or even 600° C. It is believed that such fire-climax microbes are species of Bacillus sensu lato, for example, Bacillus fusiformis, Bacillus subtilis, Bacillus megaterium, Brevibacillus centrosporus, Bacillus circulans, Bacillus simplex, Bacillus niacini, Bacillus sphaericus, Bacillus amyloliquefaciens, Bacillus insolitus, Bacillus psychrodurans, Bacillus sporothermodurans, Brevibacterium frigoritolerans, Paenibacillus azotofixans, Paenibacillus amylolyticus, Paenibacillus lautus, Brevibacterium spp., Brevibacillus spp., Paenibacillus spp, Geobacillus spp., Alicyclobacillus spp., Sulfobacillus spp., Ammoniphilus spp., and Aneurinibacillus spp.

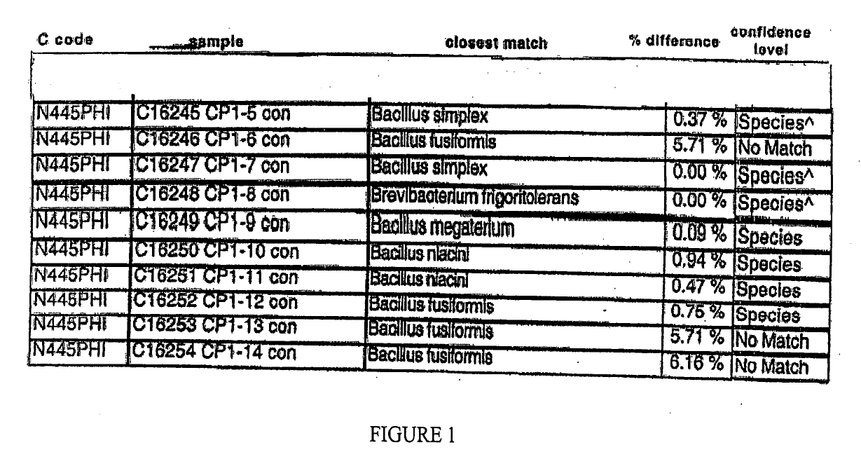

Examples of isolated fire-climax microbes include those isolated from mesquite charcoal (FIG. 1), particularly the four isolates designated as CP1-2, CP1-6, CP1-13 and CP1-14. The 16S rDNA sequences of these four isolates were not found to match a sequence in Genbank by greater than 97%.

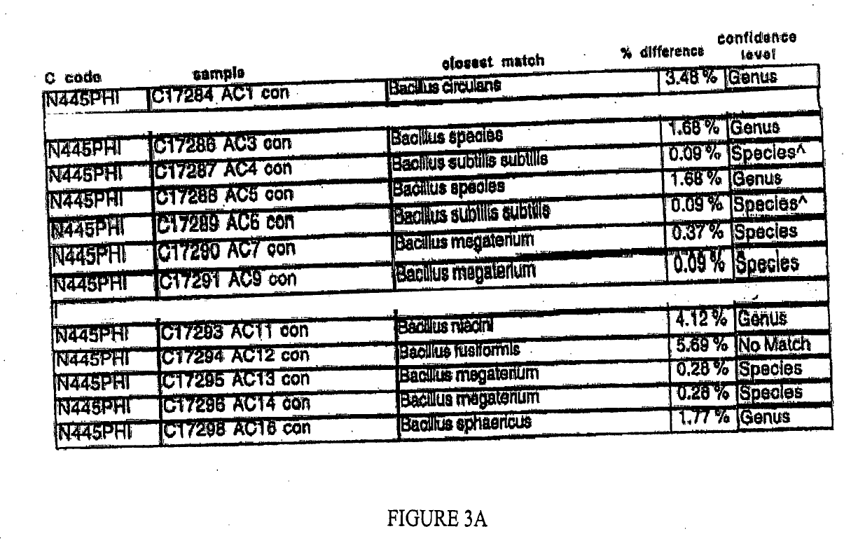

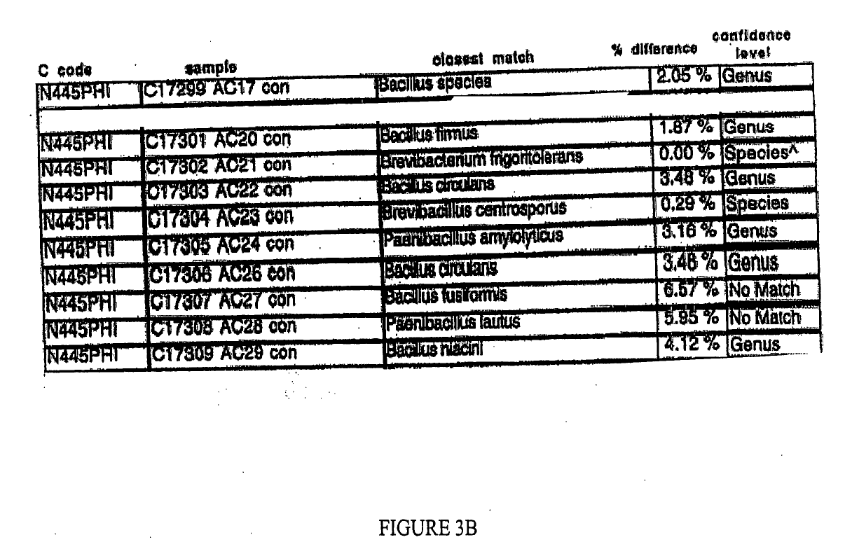

Additional examples of isolated fire-climax microbes are those isolated from almond charcoal (FIGS. 3A-3B), particularly the isolate designated as “C17304 AC23 con”. C17304 AC23 con (FIG. 3B) is determined to be Brevibacillus centrosporus and is resistant to spectinomycin, tetracycline and chloramphenicol.

Another example of an isolated fire-climax microbe is HAB7, isolated from soil. Similar to C17304 AC23 con, HAB7 is also a strain of Brevibacillus centrosporus and is resistant to spectinomycin, tetracycline and chloramphenicol. It is believed that HAB7 and C17304 AC23 con may represent one and the same isolate. The charcoal containing C17304AC23 con came from an almond tree growing in the same soil from which HAB7 was isolated. In addition, HAB7 and C17304 AC23 con are identical in their 16S rDNA sequences analyzed (500 bp). Moreover, HAB7 and C17304 AC23 con share the same antibiotic resistance, i.e., naturally resistant to a combination of spectinomycin (25 μg/mL), tetracycline (1 μg/mL) and chloramphenicol (2.5 μg/mL).

Preferred fire-climax microbes of the present invention are those that are determined to enhance plant growth and are capable of carbon fixation, for example, HAB7 and C17304 AC23 con.

Bacterial strains HAB7 and AC9 were deposited with the American Type Culture Collection, Manassas, Va. 20108, on Nov. 17, 2006.

Compositions Containing Fire-Climax Microbes

Fire-climax microbes prepared from charcoal and soil as described above can be used to prepare a composition useful for enhancing plant growth and improving soil. Therefore, the present invention provides plant fertilizer compositions and soil improving compositions that contain at least one fire-climax microbe isolated according to the methods of the present invention.

By “at least one fire-climax microbe” it is meant a single fire-climax microbe strain, or a combination of multiple (i.e., at least two) strains of fire-climax microbes. For example, a composition can include a mixture of fire-climax microbes produced by inoculating a growth medium with charcoal containing spores of fire-climax microbes and incubating the growth medium for a period of time to initiate the vegetative growth of the fire-climax microbes. It is not absolutely necessary to isolate individual strains of fire-climax microbes for use in preparing the compositions of the present invention. However, in a preferred embodiment, one or more isolated fire-climax microbe strains are employed to prepare a composition for enhancing plant growth or improving soil. Particularly preferred fire-climax microbes for use in the compositions of the present invention are those that have been determined to enhance plant growth, for example, HAB7 and AC9. In a specific embodiment, the composition includes both HAB7 and AC9.

In addition to microbes, the compositions of the present invention can include charcoal. Charcoal has been shown by the present invention to independently promote vegetative growth of fire-climax microbes, which in turn stimulate plant growth.

The compositions of the present invention can also include other components or ingredients suitable for use in fertilizer compositions.

Methods for Enhancing Plant Growth and Improving Soil:

In one embodiment, the present invention also provides a method for enhancing plant growth by employing fire-climax microbes of the present invention. Cultivars of a plant are grown in a plant cultivation medium supplemented with at least one fire-climax microbe of the present invention to achieve enhanced growth.

According to the present invention, fire-climax microbes are added to a plant cultivation medium, such as soil or a synthetic cultivation medium, in an amount effective to enhance plant growth and productivity. For example, fire-climax microbes are added to soil before, during or after planting at 2×10⁷ to 2×10¹¹ CFU/ft², or preferably 2×10⁸ to 2×10¹⁰ CFU/ft², or more preferably about 2×10⁹ CFU/ft². Fire-climax microbes can be inoculated to a cultivation medium in the form of spores or cells in any manner. appropriate, including spraying powders or liquid suspensions containing the fire-climax microbes. Those skilled in the art can readily determine the precise amount of fire-climax microbes, and the timing and frequency of the inoculation, that are effective to promote plant growth. An enhanced plant growth can be determined based on an increased shoot dry weight, seed yield, root growth, and/or fruit yield/size in comparison with plants grown in otherwise identical medium without the addition of fire-climax microbes.

According to the present invention, the growth of a variety of plants can be enhanced by practicing the present methods, including, but not limited to, grain crops (e.g. corn, wheat, and soybeans), sorghum, fruiting and nut-bearing trees (e.g., almonds), soybeans, peanuts, grapes, apples, berries (strawberries, blackberries, raspberries), tubers (e.g. potatoes, sweet potatoes), corn, cereal grains (e.g. wheat, rice, rye), tomatoes, onions, cucurbits (e.g., watermelon, cucumber, and cantaloupe), leafy vegetables (e.g., lettuce, spinach, endive), cotton and other commodity crops.

In another embodiment, cultivars of a plant are grown in a plant cultivation medium supplemented with at least one fire-climax microbe of the present invention to produce nutritionally-enhanced plant products.

By “nutritionally-enhanced plant product” it is meant that the product of a plant grown in the presence of at least one fire-climax microbe of the present invention has an increased amount of a phytochemical or nutrient in comparison to a plant product grown in the absence of the fire-climax microbe or microbes. Plant products include any part of plant that is intended for consumption, e.g., roots, stems, leaves, juice, fruit, oil or flowers.

The term “phytochemical” is a general term for non-nutrient plant substances.

Many phytochemicals and the products of their conversion after consumption have been shown to be protective against diseases. Phytochemicals include, e.g., carotenoids, antioxidants (e.g., flavonoids), and tocopherols (e.g., αand γ-tocopherol).

In a specific embodiment, the nutrient or phytochemical increased is a flavonoid or isoflavonoid. In other embodiments, the nutrient or phytochemical is a vitamin, for example, vitamin E (α-tocopherol or γ-tocopherol). The methodology of present invention can achieve an increase in the amount of α-tocopherol by at least 1.3-fold, or about 1.3-fold to 2-fold, and an increase in the amount of γ-tocopherol by at least 1.3-fold, or about 1.3-fold to 3-fold.

The methods of the present invention can be applied to increase the content of a nutrient or phytochemical in various plant products, including fruits (e.g., grapes, apples, berries such as strawberries, blackberries, and raspberries), tubers (e.g. potatoes, sweet potatoes), nuts (e.g., almonds, peanuts), soybeans, corn, cereal grains (e.g. wheat, rice, rye), tomatoes, onions, cucurbits (e.g., watermelon, cucumber, and cantaloupe), leafy vegetables (e.g., lettuce, spinach, endive). In a specific embodiment, the plant products are almonds, and the plant from which the nutritionally-enhanced products are harvested is an almond plant.

In a further embodiment, the present invention provides methods for enhancing the growth and/or nutritional values of a plant by growing cultivars of the plant in a plant cultivation medium supplemented with charcoal. In a specific embodiment, the charcoal for use in the methods of the present invention has been selected for containing at least one fire-climax microbe that stimulates plant growth.

The plants grown in the presence of microbes and the products of such plants form another embodiment of the present invention. The plants grown in the presence of fire-climax microbes are believed to have enhanced genes and DNAs, and manifest as having enhanced growth and increased production of e.g., phytochemicals. Such DNA-enhanced plants and plant products are beneficial to animals that ingest them, not only because of an enhanced nutritional value of the plants and plant products, but also because the DNA-enhanced plant materials are believed to modulate and enhance the DNAs in animals (such as humans), resulting in, e.g., an enhanced innate and adaptive immune system in the animal. Thus, in one embodiment, plant products containing the fire-climax microbes of the present invention are ingested by animals, including humans, to enhance the microbial flora, promote and improve health, and reduce illness. Without wishing to be bound by a particular mechanism or theory, the ingested fire-climax microbes are believed to inhibit or even kill disease-causing bacteria in the animals' system, thereby improving health and reducing disease and illness.

In a further embodiment, fire-climax microbes isolated in accordance with the present invention are employed to improve and reconstruct soil, and to increase biodiversity in soil. Methods for improving soil quality and the resulting, amended soils are additional embodiments of the present invention.

Soil to be amended can be inoculated with fire-climax microbes in the form of spores or cells. A single strain or a mixture of multiple strains of fire-climax microbes can be used. Alternatively, charcoal particles containing fire-climax microbes can be added to a soil directly without isolating the fire-climax microbes within the charcoal, or with only minimal amount of culturing and processing of microbes in the charcoal. Inoculation of soil can be achieved by, for example, spraying powders or liquid suspensions containing fire-climax microbes, or by spraying particles of charcoal containing fire-climax microbes.

The present invention has identified that certain fire-climax microbes present in a soil enhance the growth and productivity of plants grown in that soil; and that the fire-climax microbes live within the plants and survive within the charcoal produced from carbonization of the plants. Carbonized wood has key physical and chemical traits that contribute to ecosystem rejuvenation. In addition to storing microorganisms, carbonized wood contains diverse chemical catalysts. The nanometric parameters of carbonized wood create infinite surfaces for chemical reactions that sustain life and form innumerable chambers that trap and release gases. The result is a profound, highly evolved interface between the organic remains of plant life and the rejuvenating activity of microorganisms. One desirable trait of carbonized wood is that it contains connected aqueous and non-aqueous environments, sprinkled with newly recognized catalysts which can both supply and accept electrons at many different redox potentials. This complex, poorly understood matrix thus nourishes an intricate community of interconnected microorganisms. As one simple example, facultative anaerobic bacteria release H₂ as N₂ is reduced to ammonia in the absence of O₂, while aerobic bacteria in surrounding carbonized wood micelles can use the H₂ to reduce CO₂ while transferring electrons to O₂ and thus creating an anaerobic environment favoring N₂ fixation. Bacillus sensu lato species present in carbonized wood could fulfill all of these functions.

The values of charcoal have been uniquely recognized by the present invention. According to the present invention, charcoal can be employed as a carrier of fire-climax microbes to translocate fire-climax microbes from one ecosystem to another, and to transfer a plant-growth promoting property of a soil in one geographic location to another. For example, suitable bacteria in premium wine-growing regions can be transferred by means of charcoal to other selected geographic sites.

Soils modified in accordance with the present invention support plant growth independent of commercial fertilizers and pesticides, and do not create environmental pollution. Moreover, carbon-fixing properties of fire-climax microbes allow atmospheric carbon to be fixed in organic compounds in the soil, removing carbon dioxide from the air, and improving carbon storage in soil.

Accordingly, in a further embodiment, the fire-climax microbes of the present invention can be used to enhance solar energy conversion by plants, and to reduce environmental pollution by enhancing the efficiency of mineral nutrient utilization by plants. The basis of this increased efficiency lies in the capacity of fire-climax microbes to increase plant root growth. Larger roots provide greater access to soil water and mineral nutrients. An increase in these limiting factors enhances photosynthetic conversion of solar energy to plant biomass. Promoting plant growth by 25% on 80% of the US agricultural acreage could increase total carbon storage, and thus biomass production potential, by 20%. An additional benefit of this technology would be a reduction in the leaching of fertilizers, which often poison ground water and promote eutrophication of surface streams.

The present invention is further illustrated by the following examples.

### EXAMPLES

### Example I

### Identification of Novel Fire-Climax Bacterial Strains in Mesquite Charcoal

Cultures were grown from mesquite wood charcoal obtained by burning mesquite wood. The charcoal fines, dust-like particles of approximately 2 mm-15 mm in diameter, were separated from the larger pieces of charcoal. Bacteria were isolated from CP-1 (a source of mesquite charcoal) fines by autoclaving 250 ml of TY agar medium in a flask for 45 minutes at 15 lb/in², removing the medium from the autoclave, and while the temperature of the sterile agar was approximately 200° F., preparing a 2% charcoal suspension by adding 5 g of non-sterile CP1 “fines” to the 250 ml solution. The flask was swirled to mix the suspension and 20 ml of agar medium containing the charcoal was poured into each 90-mm Petri plate. Plates were cooled at room temperature, and the agar allowed to solidify. After 28 hours, bacterial and fungal colonies developed on the plates. After 72 hours, 14 bacterial colonies were picked and transferred to fresh TY agar plates. Twelve of the 14 colonies grew on the second set of plates. These 12 colonies were passaged again on a third set of TY agar plates. The 16S rDNA of the colonies that grew from the third passage was partially sequenced (Midi Labs, Newark, Del.).

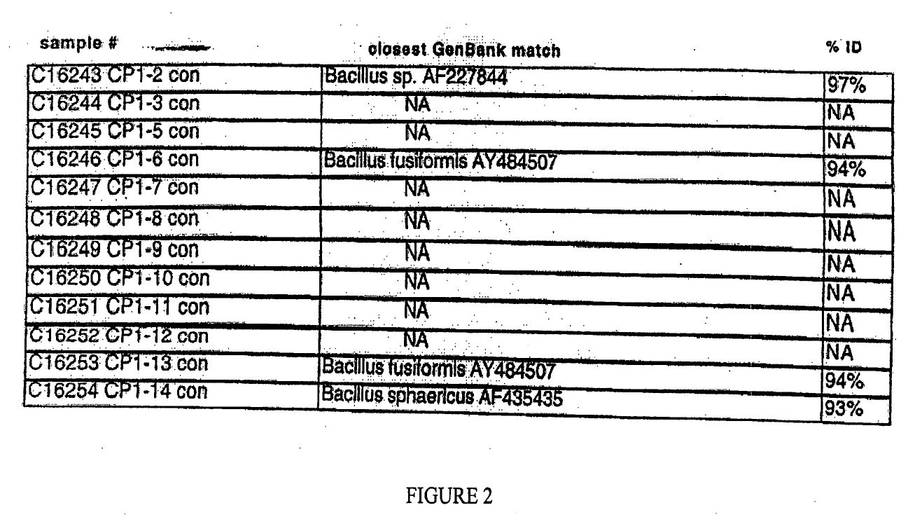

FIG. 1 shows the closest bacterial sequence matches and the corresponding percent sequence differences. Four isolates, CP1-2, CP1-6, CP1-13, and CP1-14, were not identifiable. The “closest match” designates for each of these isolates differed in sequence by >5%, thus were concluded unlikely to be the same. FIG. 2 shows the closest GenBank sequence matches found for the four unmatched isolates. The CP1-2, CP1-6, CP1-13, and CP1-14 16S rDNA sequences were not found to match any sequence in GenBank by greater than 97%.

### Example II

### Identification of Novel Fire-Climax Bacterial Strains in Almond Charcoal

The 16S rDNA sequences of twenty-nine isolates from almond charcoal were examined. Of these isolates, one was identified as Brevibacillus centrosporus. Furthermore, nineteen isolates were different, eight were identified to the species level (≦1% difference), six differed from known species by 1% to greater than or equal to 5%, and three differed from known species by greater than 5%. The sequence identification tables are shown in FIGS. 3A-3C.

Partial 16S rDNA sequence analysis showed that the almond charcoal isolate designated “C17304 AC23 con” in FIG. 3B had a 0.29% difference from Brevibacillus centrosporus, similarly to HAB7. HAB7 is a bacterial strain isolated from soil, as further described in Example III. C17304 AC23 con was also found to grow in a combination of 25 mg/L spectinomycin, 1 mg/L tetracycline, and 2.5 mg/L chloramphenicol, as was HAB7.

### Example III

### Process for Carbonizing Douglas Fir Wood and Bark Shavings and Isolation of Fire-Climax Microbes from the Carbonized Materials

A recently felled Douglas fir tree was cut into 14″ sections. Bark tissue (i.e. phloem) and wood (i.e. xylem) were shredded separately with a 20″ chain saw and collected. The two plant tissues were mixed together in a 1:1 ratio, dried overnight at 70° C., and stored in sealed glass jars for later use.

A series of 2″ diameter ceramic crucibles were loaded with 10 g of the mixed tissues. A 240V Bamstead International muffle furnace, Model F47920-80, with programmable temperature control and a 5″(W)×4″(H)×6″(D) chamber, was used for all experiments described in this Example. This furnace, which can run at 1093° C. continuously or 1200° C. intermittently, was used to mimic forest-fire conditions. The furnace was preheated to 600° C., and individual crucibles were placed in the furnace for various periods of time. When a crucible was placed in the furnace chamber, the temperature dropped briefly to about 585° C. before it returned to 600° C. After 4 min., the furnace temperature increased to 605° C. for several minutes while black smoke escaped from a top vent. Crucibles were removed after 5, 10, and 15 min., cooled and covered with aluminum foil. Two crucibles were treated separately for each time period. One was used for isolating microorganisms; the other was photographed. All experiments reported in this Example involved placing only one crucible in the furnace for the specified time period to achieve consistent carbonization.

Physical characteristics of the samples treated for various periods were quite different. Samples removed after 5 min at 600° C. were carbonized approximately 50% as estimated by the decrease in dry weight and many wood chips were unblackened. Those remaining in the furnace for 10 or 15 min were completely blackened and reduced 80-90% in mass. No chips retained a natural wood color after 10 or 15 min at 600° C.

The number of bacterial isolates obtained from samples treated for various periods differed. Bacteria were isolated by suspending each carbonized sample in 250 mL of hot sterile TY medium as it came out of the autoclave at approximately 100° C. The suspension was swirled vigorously as twelve 100-mm Petri plates were poured from each mixture. After 4 days of incubation at 20-25° C., the plates were observed and 14 of 41 bacterial colonies were picked for identification by 16s rDNA sequencing. Most of the isolates fell among the classical Bacillus-like bacteria. Rhodococcus globerulus however, is an actinomycete organism more closely related to soil microorganisms such as Streptomyces (Table 1). Numerical counts showed that 10 min at 600° C. clearly produced optimum conditions for isolating fire-climax microorganisms from wood in these experiments. Other combinations of wood and bark tissues could have different thermal characteristics, which would create an altered optimum. Other woody species may contain additional bacterial species.

As indicated, in accordance with the present invention, the physical nature of the sample itself is important because, inter alia, number, shape, size and consistency of the sample will affect how quickly external heat is transferred throughout a sample.

Moisture contained within the sample or the air surrounding the sample will also change carbonization traits because the high heat of vaporization of water uses heat energy to convert water from the liquid to the vapor phase (i.e., boil). Thus the relative percent humidity and the elevation above sea level (i.e., atmospheric pressure) can change the rate of carbonization.

Thermal settings on the furnace and time of exposure also will influence the rate of carbonization. Numerical value of a constant temperature is important. Higher temperatures will speed carbonization relative to lower temperatures. Longer time periods at lower temperatures may be equivalent to shorter periods at higher temperatures, as long as the sample burns. Programmed changes in temperature also will produce different conditions for similar states of carbonization.

TABLE 1
Colony-forming units (CFU) produced
from 10 g of Douglas fir tissues.

| | Bacterial | Fungal | |
|---|---|---|---|
| Time at | colonies | colonies | Bacterial species identified by 16S |
| 600° C. | (cfu/10 g | (cfu/10 g | rDNA (i.e. <1.00% difference from |
| (min) | tissue) | tissue) | standard strains) |
| 0 | 0 | 0 | Not applicable |
| 5 | 0 | 1 | Not applicable |
| 10 | 41 | 0 | Bacillus amyloliquefaciens, B. subtilis, |
| | | | B. pumilus, Brevibacterium |
| | | | frigoritolerans, Brevibacillus |
| | | | choshinensis, Rhodococcus globerulus |
| 15 | 0 | 0 | Not Applicable |

Bacterial species were also isolated from other forms of carbonized organic matter. A sample of commercially available fish meal, which is a form of oceanic organic waste and can be found in many organic gardening shops, was heated at 600° C. and suspended in 95° C. TY agar before bacteria were isolated and identified by partial sequencing of the 16S rDNA (Table 2). Sequences diverging more than 1% from the MicroSeq data base also were compared with the GenBank data base. Isolates recovered at different times were mutually exclusive.

TABLE 2
Isolation of Bacterial Species from Oceanic Organic Waste

| Time at | | | | | |
|---|---|---|---|---|---|
| 600° C. | Bacteria | MicroSeq ID (% difference from | | GenBank ID (% similarity to | |
| (min) | (cfu/10 g) | closest match) | | closest match) | |
| 0 | Too many to | Bacillus badius | (0.09) | Not applicable | |
| | count | Bacillus amyloliquefaciens | (0.00) | Not applicable | |
| | | Bacillus globisporus | (4.68) | Bacillus aquamarinus AF202056 | (98) |
| | | Bacillus halodenitrificans | (8.91) | Bacillus sp. AF329473 | (98) |
| | | Bacillus sporothermodurans | (1.07) | Bacillus oleronius AY988598 | (98) |
| | | Bacillus sp. | (5.34) | Sporosarcina sp. AJ971924 | (97) |
| 5 | Too many to | None sequenced | | — | |
| | count | | | | |
| 10 | 6 | Bacillus niacini | (5.08) | Bacillus arbutinivorans AF519469 | (98) |
| | | Bacillus smithii | (8.57) | Bacillus endophyticus AY211143 | (99) |
| | | Bacillus niacini | (3.09) | Bacillus sp. DQ275174 | (98) |
| 15 | 6 | Bacillus niacini | (3.18) | Bacillus sp. DQ275174 | (98) |
| | | Bacillus chitinolyticus | (8.18) | Paenibacillus sepulori DQ291142 | (95) |

### Example IV

### Culturing of Fire-Climax Microbes from Soil

Cultures were grown from sandy loam soil samples (Mid-Cal Ranch, Calif.) to identify the microbes capable of sporulation present in the samples. For this purpose, 2.0 ml of a solution created by suspending equal volumes of soil and water were placed in a. boiling water bath for 3 minutes before plating an aliquot (e.g. 0.5 ml) on agar TY medium (Difco Bacto tryptone (5 g/1), Difco Bacto Yeast Extract (3 g/l) and CaCl₂ (1.3 g/l), with Difco Bacto Agar (15 g/l)).

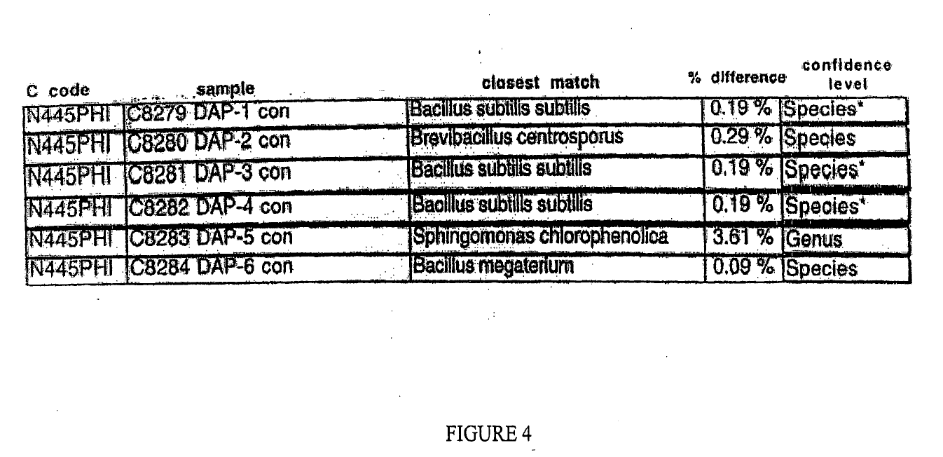

Bacteria initially isolated by these procedures were identified, by 500 base pair 16S rDNA sequencing, as aerobic endospore-forming bacterial species. These isolates were identified as Brevibacillus centrosporus, Bacillus subtilis, and Bacillus megaterium (Midi Labs, Newark, Del.). FIG. 4 shows, for each of samples DAP-1 to DAP-6, the closest bacterial sequence match and the corresponding percent difference in sequence between the sample strain and the closest match. DAP-2 (also referred to herein as HAB7) was determined to be Brevibacillus centrosporus.

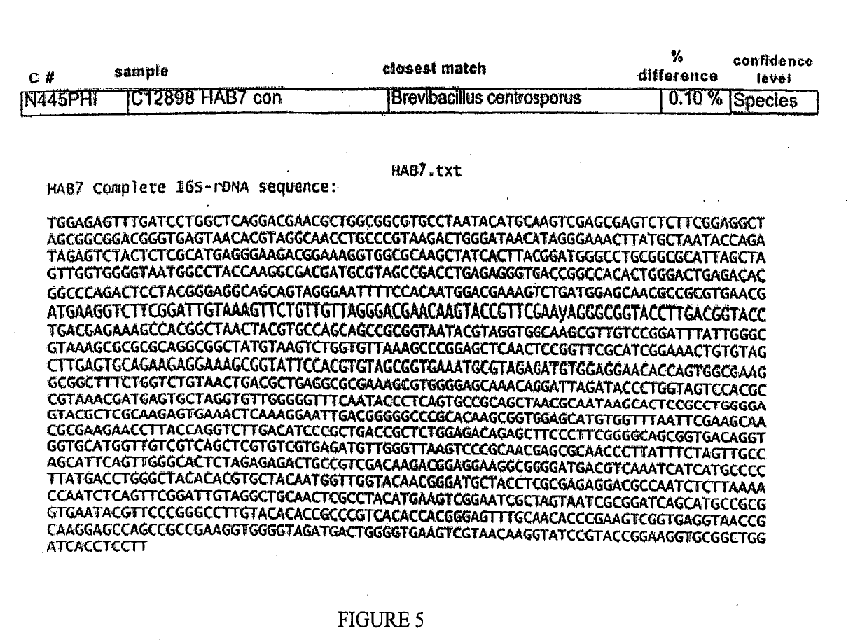

HAB7 was found to have resistance to multiple antibiotics, and to be capable of growth on a rich agar medium (TY) in the combined presence of 25 mg/L spectinomycin, 1 mg/L tetracycline, and 2.5 mg/L chloramphenicol. Based on the partial 16S rDNA sequencing, HAB7 was found to have a 0.29% difference from Brevibacillus centrosporus, indicating a species match. The complete 16S-rDNA sequence obtained for HAB7, further indicating a 0.10% difference from Brevibacillus centrosporus, is provided as FIG. 5.

### Example IV

### Effect of Bacterial Strain HAB7 and AC9 on Plant Growth

The ability of HAB7 to promote total plant growth was evaluated in two studies. In the first study, wheat seed was planted in field plots using different soil treatments, including combinations of high (100-11.25-15 lb/ac) and low (30-11.5-15 lb/ac) NPK, with and without HAB7 at 2×10⁹ CFU/ft². The soil was treated two days after planting, and harvested four months after planting. Three adjacent plots, each 3 feet by 5 feet, were cultivated under each of the five conditions specified in Table 3. Therefore, a total of fifteen plots were used, and each number shown in the table represents the mean value from three plots subjected to the same treatment. An area 1×3 ft² was harvested in the center of each plot. HAB7 was found to increase seed yield significantly in the presence of either high or low NPK. Root growth was not measured. The treatments and results are shown in Table 3.

TABLE 3

| | Shoot | | | | N | Protein | Total N |
|---|---|---|---|---|---|---|---|
| | Dry Wt | | Seed Yield | | Content | Content | Harvested |
| Treatment | (g/3 ft²) | | (g/3 ft²) | | (%) | (%) | (mg) |
| Untreated Control | 230 | | 46 | | 1.57 | 9.81 | 722 |
| Low NPK | 270 | | 63 | | 1.39 | 8.69 | 876 |
| (30-11.25-15 lb/ac) | | | | | | | |
| High NPK | 375 | | 94 | | 1.48 | 9.25 | 1391 |
| (100-11.25-15 lb/ac) | | | | | | | |
| Low NPK + | 292 | (+8%) | 87 | (+38%)* | 1.41 | 8.81 | 1227 |
| HAB7 | | | | | | | |
| High NPK + | 437 | (+16%)* | 127 | (+35%)* | 1.67 | 10.44 | 2121 |
| HAB7 | | | | | | | |

| | | |
|---|---|---|
| LSD 0.05^{#} | 53 | 17 |

*HAB7 effect was significant (P < 0.05) relative to the fertilizer treatment alone.
^{#}LSD 0.05 values measure significant differences across all treatments.

As shown in Table 3, the addition of HAB7 to low NPK resulted in an 8% increase in shoot dry weight and a 38% increase in seed yield. The addition of HAB7 to high NPK resulted in a 16% increase in shoot dry weight and a 35% increase in seed yield. A large increase in nitrogen was also measured in the wheat plants grown in HAB7/high NPK-treated soil.

Root growth, most accurately evaluated in pots from which all roots are recoverable, was measured in a second study by growing annual rye grass from seed in the presence or absence of HAB7, with either high or low NPK. The grass was planted in 64 ten-inch pots. HAB7 cells were supplied at 2×10⁹ CFU/ft² to each HAB7 treatment pot. The grass was harvested 28 days following planting, before the roots became pot-bound. The plants were harvested prior to flowering, therefore seed yield measurements were not made. After drying the plants, all sand, pebbles and debris were removed, and the plants were separated into shoots and roots. The treatments and results of this experiment are shown in Table 4. The table shows that root growth was increased by 11% (P≦0.05) in the presence of HAB7, and by 33% (p<0.05) when a combined HAB7/high NPK treatment was provided.

TABLE 4

| Treatment | Shoot (g) | | Root (g) | | Total (g) | |
|---|---|---|---|---|---|---|
| None | 2.52 | ab | 2.67 | b | 5.19 | ab |
| NPK (30-11.25- | 2.85 | ab | 2.16 | c (−19%) | 5.00 | b |
| 15 lb/ac) | | | | | | |
| HAB7 (no NPK) | 2.40 | b | 2.95 | a (+11%) | 5.35 | ab |
| NPK + HAB7 | 3.06 | a | 2.88 | ab (+33%) | 5.95 | a (+19%) |

* “NPK” represents nitrogen, phosphorus and potassium. Values followed by different letters showed significant effects of HAB7 (p < 0.05) relative to the appropriate control.

AC9, as well as a mixture of HAB7 and AC9, were also shown to promote plant growth in a nutrient-rich potting soil. Representative data from two tests are given in Table 5. Variation in uninoculated control plants reflects changing temperature and light in different months of the year.

TABLE 5
Effects Of Bacterial Inoculants On Wheat Growth After 28 Days

| Treatment | Wheat Shoot Dry Weight (mg/plant) | |
|---|---|---|
| Uninoculated | 535 ± 30 | 219 ± 11 |
| AC9 | 662 ± 45 (+24%) p ≦ 0.01 | |
| HAB7 + AC9 | | 262 ± 13 (+ 20%) p ≦ 0.01 |

### Example VI

### Effect of Charcoal on Bacterial Growth

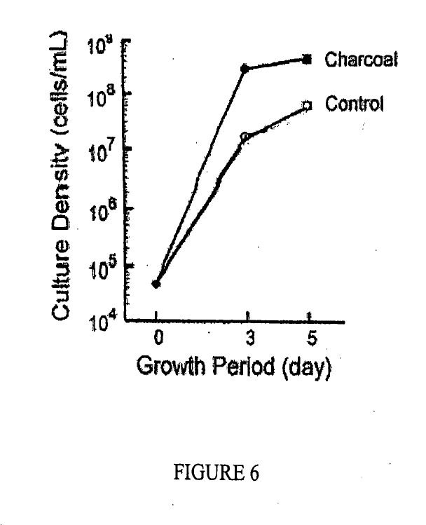

The promotive effects of charcoal on the growth of a representative microbe was easily demonstrated with HAB7. HAB7 was grown in TY bacterial medium containing either no additive, or 1.5% crushed mesquite charcoal (“CP-1”). All media were adjusted to pH 6.74 before inoculation. Cells were counted as colony-forming units (CFU) on TY agar plates with normal dilution techniques on Days 0, 3 and 5 days following inoculation. Cell density just after inoculation was 5×10⁴ CFU/ml. The results show that the charcoal had enhanced bacterial growth approximately 20-fold on Day 3 and 8-fold on Day 5 as compared with untreated control cultures (FIG. 6). The Day 5 effect, determined using three replicate cultures for each treatment, was highly significant (P≦0.01). The Day 3 effect was measured with a single replicate in each treatment.

An experiment performed similarly in which lower (0.375%) and higher (3%) concentrations of crushed charcoal were tested showed that 1.5% charcoal enhanced bacterial growth over untreated control cultures, whereas the lower concentration had no significant effect on growth but reduced sporulation significantly. The 3% charcoal treatment had a greater enhancing effect on bacterial growth than did the 1.5% charcoal, and resulted in fewer spores. The data measured 3 days after inoculation are shown in Table 6. Colonies developing from spores, rather than from vegetative cells, were identified by their delayed development and smaller colony size on day 3.

TABLE 6

| | Total Bacteria (CFU/ml) | |
|---|---|---|
| Treatment | Mean ± Std Error | Colonies from spores (%) |
| Untreated | 4.76 ± 0.7 × 10⁷ | 14.4 ± 1.2 |
| Charcoal, 0.375% | 3.34 ± 0.2 × 10⁷ | 9.2 ± 0.4 |
| Charcoal, 1.5% | 13.0 ± 0.2 × 10⁷ | 4.6 ± 0.1 |
| Charcoal, 3.0% | 16.1 ± 0.8 × 10⁷ | 2.0 ± 0.5 |

| | | |
|---|---|---|
| Statistical Comparison: | Total Bacteria | % Colonies from spores |
| 1.5 & 3.0% vs TY | P < 0.001 | P < 0.01 |
| 0.375% vs TY | Not significant | P < 0.05 |
| 1.5 vs 3.0% | P < 0.05 | P < 0.01 |

### Example VII

### Carbon Fixation by HAB7

The ability of HAB7 to incorporate ¹³C from ¹³CO₂ was examined. Nine 50-ml Erlenmeyer flasks, each containing 25 ml of sterile TY liquid medium were supplied with a 2% inoculum of HAB7 from an overnight culture and grown for 36 hours. Cultures were placed on a shaker at 145 rpm at 25° C. All flasks were sealed with rubber septa. Three replicate flasks contained atmospheric CO₂ with approximately 0.042% CO₂ (1.085 atom % ¹³C). Six replicate flasks were supplied with 5% ¹³CO₂ generated from ¹³C-sodium bicarbonate (98 atom % ¹³C) in a 60-cc syringe with 2N HCl. Five ml aliquots were collected from each flask after 12, 18 and 36 hours and bacterial cells were removed by centrifugation in Eppendorf tubes. Flasks were opened to air and resealed in a sterile transfer hood, and those designated for 5% CO₂ were replenished with 5% ¹³CO₂ at each time of sampling. Samples in Eppendorf tubes were freeze-dried and analyzed for ¹³C content by ¹²C/¹³C isotope ratio mass spectrometry. The change in ¹³C relative to a standard reference 1.08504 atom % ¹³C was calculated and reported as Δ¹³C atom %. The possibility of contamination of HAB7 was ruled out at the end of the experiment by comparing cell dilution counts on normal TY medium with counts on TY containing a mixture of three antibiotics selective for HAB7. Data (in CFU) were similar on the two media for each flask.

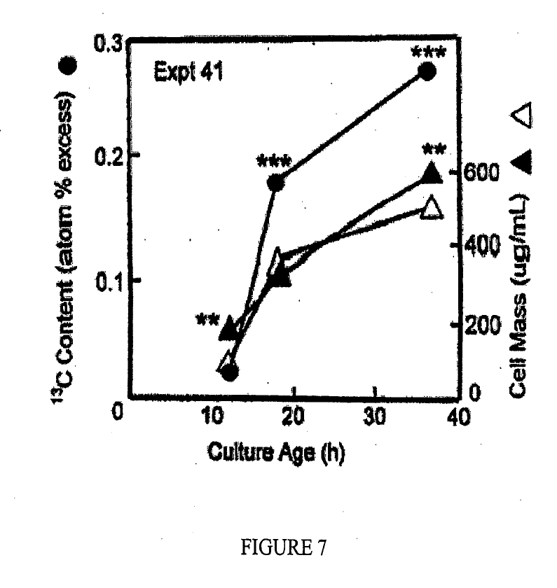

As shown in FIG. 7, HAB7 cells incorporated CO₂ to a level of approximately 0.25% of total carbon (black circles). Increases in ¹³C content between 12 and 18 hours and between 12 and 36 hours were highly significant (p≦0.001). Increased CO₂ concentration (black triangles) promoted cell growth (mass) by 50% at T₁₂ (12 hour) (P≦0.01) and by 12% at T₃₆ (36 hour) (P≦01.01) relative to ambient atmospheric CO₂ (open triangles).

### Example VIII

### Carbon Fixation by Fire-Climax Microbes in Charcoal

Tests established that fire-climax microbes present in mesquite charcoal (CP-1) clearly incorporated CO₂ (Table 4). Erlenmeyer flasks (50-ml size), each containing 25 ml of sterile TY liquid medium, were inoculated with 1) autoclaved CP-1 in the presence of ambient CO₂ to quantify any physical effects of charcoal on adsorption of background ¹³C, or 2) autoclaved CP-1 in the presence of 5% ¹³CO₂ to quantify any physical effects of charcoal on adsorption of ¹³C from the highly labeled ¹³CO₂ (98 atom % ¹³C), or 3) non-sterile CP-1 in the presence of 5% ¹³CO₂ to quantify any effects of microorganisms in CP-1 on the uptake of CO₂ from the highly labeled ¹³CO₂ (98 atom % ¹³C), or 4) sterile CP-1 inoculated with HAB7 in the presence of 5% 1³CO₂ to relate the proven capacity of these bacteria to take up CO₂ to other treatments in this test. Thus CO₂ levels were ambient 0.42% CO₂ (1.08515 atom % ¹³C) or 5% CO₂ (98 atom % ¹³C). Flasks remained sealed for the entire 72-hour experiment. Samples from flasks in treatments 1 and 2 were plated on TY medium at the end of the study to confirm sterility before ¹³C analyses. Flasks in treatments 3 and 4 were autoclaved at the end of the 72-hour period and then charcoal was separated by filtration from the bacterial suspension in each of these flasks. The charcoal was dried at 75° C. before ¹³C analyses. Bacteria (and some CP-1) in treatments 3 and 4 were collected by centrifugation of the bacterial suspension and dried under a heat lamp before ¹³C analyses. The ¹³C content of each sample was measured by ¹²C/¹³C isotope ratio mass spectrometry. The change in ¹³C relative to a standard reference containing 1.08504 atom ¹³C was calculated and reported as Δ¹³C atom %.

The data indicate that the CP-1 microorganisms incorporated CO₂ to levels that were significantly greater than the sterile ¹³C control (P<0.01) and were similar to those measured in the HAB7 treatment, an organism demonstrated to incorporate CO₂ in Example VII.

TABLE 7

| Treatment | | Fraction | ¹³CO₂ | Δ¹³C atom % |
|---|---|---|---|---|
| 1) | Sterile | Sterile charcoal | None | −0.00240 |
| | Ambient | | | |
| | Control | | | |
| 2) | Sterile ¹³C | Sterile charcoal | 5% | 0.00049 ± 0.00044 |
| | Control | | | |
| 3) | CP1 Flora | Nonsterile charcoal | 5% | 0.00470 ± 0.00233 |
| | | Bacterial filtrate | 5% | 0.145 ± 0.069 |
| 4) | HAB7 | Sterile charcoal | 5% | 0.00426 |
| | inoculum | Bacterial filtrate | 5% | 0.186 |

### Example IX

### Promotion of HAB7 Growth by Carbon Dioxide

The effect of CO₂ on the growth of HAB7 was evaluated. An overnight culture of HAB7 growing in TY medium was used to supply a 2% inoculum in 50 ml flasks, each containing 25 ml of TY and 30 ml of atmosphere above the liquid. Colony-forming units (CFU) were counted by dilution plating on TY agar medium at To (time of inoculation), T₁₅ (15 hours post-inoculation), T₃₆ (36 hours post-inoculation), and T₄₂ (42 hours post-inoculation). After inoculation and after each sampling, atmospheric CO₂ in the air space above the liquid was adjusted to contain atmospheric (approximately 0.042%), 5% CO₂, or 9.8% CO₂ on a volume basis. Cell yields were weighed at T₄₂ after collecting cells by centrifugation. The data are shown in Table 8. The 5% and 9.8% CO₂ treatments increased the CFU/ml significantly during log growth (T₁₅), but 9.8% CO₂ decreased the CFU/ml markedly at the 42 hour sampling time.

These results can be interpreted as showing that additional CO₂ stimulated fatty acid synthesis and membrane formation during log growth, but the highest CO₂ treatment eventually increased cell mortality. The extra membranes were used to increase cell numbers in flasks supplied with extra CO₂ at the beginning of the experiment. Because the TY medium was so rich, there was no substrate limitation, and thus the control treatment caught up to the elevated CO₂ flasks when cellular respiration produced enough CO₂ to support optimum membrane synthesis. Dry weight data suggest that cells in the control treatment were producing more polysaccharide than cultures grown with higher CO₂.

TABLE 8

| | Control | 5% CO₂ | 9.8% CO₂ |
|---|---|---|---|
| Time (h) | CFU/mL | CFU/mL | CFU/mL |
| 0 | 1.14 × 10⁶ ± | 1.14 × 10⁶ ± | 1.14 × 10⁶ ± |
| | 0.03 × 10⁶ | 0.03 × 10⁶ | 0.03 × 10⁶ |
| 15 | 2.64 × 10⁷ ± | 5.50 × 10⁷ * ± | 5.82 × 10⁷ * ± |
| | 3.12 × 10⁶ | 8.40 × 10⁶ | 9.45 × 10⁶ |
| 36 | 4.21 × 10⁸ ± | 3.39 × 10⁸ ± | 3.80 × 10⁸ ± |
| | 5.68 × 10⁷ | 1.36 × 10⁸ | 4.57 × 10⁷ |
| 42 | 5.17 × 10⁷ ± | 1.15 × 10⁸ ± | 6.73 × 10⁶ * ± |
| | 1.15 × 10⁷ | 4.86 × 10⁷ | 5.84 × 10⁶ |
| 42 h | 733 ± 49 | 547 * ± 46 | 597 * ± 32 |
| Dry Matter | | | |
| μg/mL | | | |

* Significantly different from the control at P ≦ 0.05.

### Example X

### Pedogenesis Regulators Increase Plant Vitamin E Content

Levels of Vitamin E (α- and γ-tocopherols) were assayed, in plant products obtained from conventionally-grown plants and plants grown in soil treated with charcoal, to evaluate the effect the compositions and methods of the invention on plant nutritional properties.

Two almond nut samples (three replicates of each) were analyzed. One sample was the commercial brand “Diamond of California,” designated “Diamond” in the data table. The second sample was obtained from- bulked almonds harvested from trees grown at Mid-Cal Ranch (“Mid-Cal”) in soil that had been treated with commercial charcoal prepared as described in Example I at a rate of 200 pounds per acre in each of the previous two years. Almond samples (6 ounces per sample) were pulverized in a coffee grinder and submitted to a commercial lab (Analytical Laboratories in Anaheim, Inc., Anaheim, Calif.) for tocopherol content analysis. The almond “paste” was extracted with methanol, and tocopherols were separated using HPLC, and analyzed using LC-MS (liquid chromatography-mass spectrometry) methods and compared with standards.

The tocopherol levels observed in each sample are shown in Table 9. The α-tocopherol content of Mid-Cal Ranch almonds was 39% (P≦0.05) higher than that of the Diamond almonds. The γ-tocopherol content of Mid-Cal Ranch almonds was 200% (P≦0.01) higher than that of Diamond almonds.

Other methods useful for evaluating tocopherol content in plant products have been described, e.g., by Gutierrez et al., 1999, “Influence of ecological cultivation on virgin olive oil quality,” J Amer. Oil Chemists Soc. 76: 617-621 and Martinez, J. M. et al., 1975, “Report About the Use of Abencor Yields Analyser,” Grasas Aceites 26: 379-385.

TABLE 9

| | Mid-Cal | Mid-Cal | Diamond | Diamond |
|---|---|---|---|---|
| | α-tocopherol | γ-tocopherol | α-tocopherol | γ-tocopherol |
| | mg/kg | mg/kg | mg/kg | mg/kg |
| Replicate 1 | 164 | 1.35 | 122 | 0.391 |
| Replicate 2 | 207 | 1.73 | 130 | 0.561 |
| Replicate 3 | 183 | 1.63 | 147 | 0.567 |
| Mean | 184.7 | 1.57 | 133.0 | 0.51 |
| Std Error | 12.4 | 0.11 | 7.4 | 0.06 |

### Example XI

### HAB7 Increased Phytochemical Content of Lettuce

Buttercrunch lettuce was planted in a Caspar greenhouse using 2-gal pots containing Ace Potting Soil. Four of the eight pots were inoculated with HAB7. Each pot was thinned to two plants after germination. Plants were harvested in about two months, weighed, and cooled on ice or under refrigeration while being transported to a lab for analysis. Equivalent-size heads of lettuce were selected from each treatment for vitamin analyses. All data were examined with two-way analysis of variance using raw values and log-transformed numbers.

The results showed that total biomass harvested (two heads/pot) did not differ significantly between the control and the HAB7 treatment. The larger head of lettuce in each pot was used for measuring vitamin content, and mean values (±SE) for those heads were 163±17 and 159±16 for the control and HAB7-treated pots, respectively.

Vitamin C content of lettuce grown in HAB7-treated pots was increased more than 50% and the total vitamin E content (α- and γ-tocopherol) was increased by more than 80%.

| | Vitamin C | Total Vitamin E |
|---|---|---|
| Treatment | (mg/100 g) | (μg/100 g) |
| None | 8.5 | 37 |
| HAB7 | 13.3 (+56%) | 67.3 (+82%) |
| | P ≦ 0.05 | P ≦ 0.05 |

### Example XII

### Effect of Urea on HAB7

The effect of urea on growth and survival of HAB7 was tested by culturing HAB7 in the presence of urea.

Based on the conventional farming practice of applying 200 pounds of nitrogen per acre, 100 mM urea was calculated as a concentration likely to be transiently present in the top one inch of a normal soil. HAB7 was inoculated into TY medium and grown overnight at room temperature to approximately 1×10⁷ CPU/ml.

The overnight culture was inoculated into flasks (200 μl overnight culture per flask) containing either TY (3 flasks) or TY+100 mM urea (3 flasks). Thirty hours later, 10-fold dilution series were prepared from each of the six flasks, and one 50-μl aliquot from each dilution tube was plated onto TY agar. Colony forming units (CFU) developing from the 50-μl drops were counted and used to calculate the number of viable cells in each flask. Mean values ± standard errors were compared for the urea treatment effects using Student's test

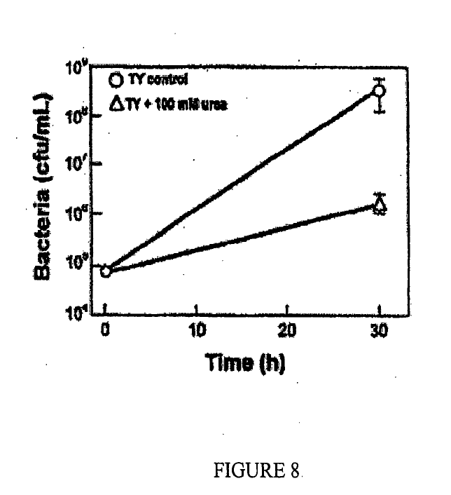

FIG. 8 shows the increase in bacterial density after thirty hours in the presence or absence of urea. In the TY, the bacteria passed through more than 12 doublings, but in the presence of 100 mM urea, fewer than 5 doublings occurred. As a result, the urea-containing flasks had less than 1% as many HAB7 cells as the control flasks. The effect of urea in this test (t=2.91, n=4) was significant (P≦0.05).

Simultaneously with the flask dilutions, a 10-fold dilution series was prepared from the overnight culture itself. Three independent aliquots of 50 μl from various dilutions of the overnight culture were plated onto separate agar plates containing either TY or TY+100 mM urea.

The plating experiment showed that 58% of the cells from the overnight culture failed to grow on the TY agar+100 mM urea plates. For example, the 10⁻⁴ dilution plates produced 50.0±5.5 colonies on TY and 21.3±3.2 colonies on TY+100 mM urea. The effect of urea in this test (t=4.51, n=4) was significant (P≦0.05).

Thus, urea at concentrations used in common farming practices adversely affects growth of HAB7 bacteria.

### Example XIIII

### Effect of Pedogenesis Regulators on Plant Flavonoid Content

The microbial organisms and carbon sources disclosed herein can also be used to increase plant phytochemical content, in particular isoflavonoids, such as phytoestrogens, and other flavonoids. Plants can be grown in soil amended with a carbon source, such as mesquite charcoal. The soil can then be amended with a microbial organism, such as Bacillus centrosporus, Bacillus subtilis, or Bacillus megaterium. A combination of microbial organisms may also be used to nucleate pedogenesis in the soil. After visible growth, plant roots and plant leafy matter are analyzed for phytochemical content, including isoflavonoid and flavonoid content.

Flavonoid content can be determined as described by Ren, H., et al., 2001, “Antioxidative and antimutagenic activities and polyphenol content of pesticide-free and organically cultivated green vegetables using water-soluble chitosan as a soil modifier and leaf surface spray,” J. Sci. Food Agric. 81: 1426-1432. Harvested vegetables grown in soil or plant cultivation medium prepared according to the methods of the invention are transported in a refrigerated container to the laboratory for analysis and comparison with reference sample vegetables grown in unamended soil. A part of the vegetable body-(50-60 kg each) can be randomly cut out from 5-20 samples (1.5-2.0 kg in total for each vegetable) of each variety to eliminate individual variation. The samples are washed with running tap water and pure water, wiped with paper towels and treated with a juicer (model MJ-C29, Matsushita Electric Co., Kobe, Japan). Juices are centrifuged at 3800×g and then 13500×g for 10 minutes each at 5° C. to remove fine particles. The supernatants are filtered through a radiation-sterilised membrane (pore size 0.45 μm, Toyo Advantec, Tokyo, Japan) for microbiological assay. All samples are stored in sterilized vials at −80° C. until analyzed for polyphenol content.

Total polyphenol and orthodiphenols can be determined by colorimetry using the Folin-Deni reagent or ammonium molybdate as described by Vazquez, A., et al., 1973, “Determinacion de los Polifenoles Totales del Aceite de Oliva,” Grasas Aceites 24:350-357. Individual flavonoid levels can be measured using a QP-8000α(Shimadzu, Kyoto, Japan) instrument for liquid chromatography/mass spectrometry (LC/MS) in combination with an STR ODS-II semi-micro column (150 mm×2.1 mm id, Shinwa Chemical Industry, Kyoto, Japan). As mobile phase, 0.2% acetic acid solution (A) and methanol (B) can be passed through the column with a gradient curve of (1) 30-50% B (0-5 mm), (2) 50-90% B (5-10 mm), (3) 90% B (10-15 mm), (4) 30% B (15-25 mm).

Pure reagents (e.g., Caffeic acid, hesperidin, hesperitin, myricetin, quercitrin, quercetin, apigenin and baicalein), for use as calibration standards are available commercially, e.g., from Sigma Chemical (St. Louis, Mo, USA). They are dissolved individually in methanol, then made up to appropriate concentration in 30% methanol. Chromatography can be carried out at a constant flow rate of 0.2 ml min⁻¹. The sample volume injected is 5 μl and the column is kept at 40° C. in the oven. Atmospheric pressure chemical ionization (APCI) is employed for the interface, and nitrogen flow is adjusted to 2.5 liters min⁻¹ as nebulizer gas. After each compound is identified by its retention time in comparison with the standard and by molecular weight information- obtained from the MS detector, quantitative determination is carried out using the one-point absolute calibration curve obtained by selected ion monitoring.

Test samples are prepared from vegetables harvested independently in at least three different months. Each test is repeated twice using vegetable juices prepared in the three different months, and the six sets of data obtained are used to evaluate the biological activities and chemical composition of each sample. Statistical significance of differences between the test samples and reference samples can be determined using a Student's t-test.

### Example XIV

### Effect of Plant Nutrients, Phytochemicals, and Bacterial Antigens on Innate Immune System Activity

The microbial antigens, plant nutrients and phytochemicals isolated from plants grown in the methods and compositions disclosed herein can also be used to increase immune system activity in mammalian species. For example, microbial antigens or nutrients and phytochemicals, including isoflavonoids and flavonoids, can be isolated from plant root or plant matter samples and fed to rats or mice. Alternatively, plant extracts can be prepared from plants grown using the methods and compositions disclosed herein, and fed to rats or mice. The effect can be determined by monitoring immune system activity, including activation of the innate immune system through the increased production of IgA, or the stimulation of toll-like receptors in the innate immune system. Innate immunity can be assessed by measurement of immunity proteins, including lysozyme and lactoferrin, in the blood of a subject (Bard, E., et al., 2003, Feb, Clin. Chem. Lab. Med. 41(2): 127-33).

Blood samples can be collected in dry tubes, immediately placed on ice and cleared by centrifuigation at 1000×g at 4° C. for 10 minutes. Aprotinin (0.0025%) and sodium azide (0.1%) are added to the supematant and aliquots of 200 μl are stored at −20° C. until use.

Saliva samples can be obtained by placing a “Salivette” (Sarstedt, Orsay, France) between gum and cheek in the axis of the ostium Stenon canal for five minutes in one side of the mouth and then in the other. The obtained saliva is immediately placed on ice, and cleared by centrifugation at 1000×g at 4° C. for 10 mm, to separate saliva from the “Salivette.” Aprotinin (0.0025%) and sodium azide (0.1%) are added to the supernatant and aliquots of 200 μl are stored at −20° C. until use.

Stool samples can be collected in 1-liter plastic jars and refrigerated at 4° C. before their transfer to the laboratory within 2 hours following collection. Fresh weight of stools is measured to determine the fecal output of proteins. Fecal protein concentrations were determined using three-fold diluted samples. To extract fecal proteins, 5 g of homogenized stools are strongly shaken with a magnetic agitator, with 10 ml of NaCl (0.15 M) at 4° C. for 1 h. After centrifugation at 3000×g for 10 minutes at 4° C., sodium azide (fecal concentration 0.1% weight/volume) and phenylmethylsulfonylfluoride (PMSF) (final concentration at 5 mM) are added to fecal extract. Sodium azide is an inhibitor of microbial proliferation and PMSF is an inhibitor of digestive enzymes (trypsin, pepsin) and bacteria. Fecal extract is then distributed in 200 μl tubes and frozen at −20° C.

Cervico-vaginal secretions can be obtained using a lavage technique described by Belec, et al. Three milliliters of sterile phosphate-buffered saline (PBS) is placed in the vagina with a pipette. After flux and reflux cycles lasting 60 seconds each, 2 to 3 ml of fluid are collected. This sample is centrifuged at 1000×g at 4° C. for 10 minutes. The supernatant is used to measure Lz and Lf while the pellet containing cells and other mucus is frozen for later study. The possibility of blood contamination can be eliminated by a Hem Check 1 test. A dilution factor often can be used for cervicovaginal lavage.

The measurements of Lz and Lf are assayed by time-resolved inumunofluorometric assay (TR-IFMA) in the collected serum, saliva, stool and cervicovaginal secretions. Microwell Immuno Plates (Microwell Maxisorp, Life Technologie, France) are coated and incubated overnight at 4° C. with purified IgG to human Lz at 5 mg/l concentration (rabbit anti-human Lz purified polyclonal JgG, Dako, Dakopans, Copenhagen, Denmark), to human Lf at 5 mg/l concentration (rabbit anti-human Lf purified polyclonal IgG, Dako) in K2HPO4 buffer (50 mmol/l, pH 8.5). Nonspecific protein-binding sites are blocked by incubation of plates with blocking solution (50 mmol/l Na2HPO4, 1% bovine serum albumin (BSA)). Serial dilutions (ratio 2.5) of test samples and standard purified Lz (seven levels: 1.02; 2.55; 4; 6.4; 10; 16 and 25 μg/l) or Lf (eight levels: 1.02; 2.55; 6.4; 10; 16; 40 and 100 μg/l) (Human Milk Lz, Sigma, Bourgoin Jallieu, France; Human Milk Lf, Sigma, Bourgoin Jallieu) are added to the wells. Blank and positive controls (Human Milk Lz, Human Milk LU at three different concentrations (in standard test. linearity) are systematically added for each plate used. The plates are incubated for 2 hours at laboratory temperature under smooth agitation. They are washed six times with an automatic plate washer and then incubated for 2 hours under smooth agitation with biotin conjugated IgG at 250 μg/l rabbit anti-human Lz biotin concentration or 250 μg/l of rabbit anti-human Lf/biotin concentration. Streptavidin conjugated with europium at 100 μg/l concentration (Streptavidin labeled with europium, Delfia™, Wallac, Turku, Finland) is added to the wells and incubated for 2 hours under smooth agitation at room temperature. After six washings with an automatic plate washer, the reaction is begun by adding enhancement solution (Delfia, Ref. 1244-104, Wallac, Turku, Finland) for 10 minutes under agitation. Fluorometric signals are read at 615 nm with a Victor 2 fluorometer (Wallac 1420 Multilabel counter, Turku, Finland). Data are analyzed by software (Multicalc 2000™, Wallac, Turku, Finland). Quantitative results are determined by reference to the standard curves. Concentrations are corrected by dilution factors taking into. account dilutions for the TR-IFMA technique.

The relative coefficient of excretion (RCE) for each protein can be determined to compare the parameters of protein excretion in human excretions, and to enable comparison with other species. The RCE expresses a protein excretion rate relative to that of albumin, which is entirely derived from plasma by passive diffusion. The RCE referring to albumin is only applicable to fluids where albumin is not degraded by enzymes, i.e., saliva and cervico-vaginal secretions. Therefore, RCE referring to AAT was used in stool samples. RCE are obtained according to the following formula: [(albumin or AAT in serum)/(albumin or AAT in fluid)]/[(protein in serum)/(protein in fluid)]. The limit (cut-off point) between secretion and transudation is equal to one (RCE of albumin or AAT). Under this limit, transudation from the serum compartment occurs, whereas RCE greater than one demonstrates a predominant local secretion with a small amount of transudation.

Results can be expressed as mean±standard error of the mean (SEM), median and range values. Comparison of means between secretions is established by the Mann-Whitney U-test. A significant difference can set at p equal to, or less, than 0.05. Statistical analyses are performed using StatViewTM software for PC (SAS Institute Inc.).

What is claimed is:

1. A method of producing a carbonized organic material suitable for isolation of fire-climax microbes, comprising heating an organic material containing spores of said fire-climax microbes under conditions such that carbonization progresses to the extent just prior to extinction of said spores of said fire-climax microbes.

2. The method of claim 1, wherein the heating is conducted under conditions about 1BTU away from the conditions that would have resulted in extinction of said spores of said fire-climax microbes.

3. The method of claim 2, wherein the heating is conducted at about 600° C. for about 10 to about 15 minutes.

4. The method of claim 1, wherein said organic material is a plant material or an oceanic waste material.

5. The method of claim 1, further comprising recovering said spores of said fire-climax microbes from the produced carbonized material.

6. A method of producing fire-climax microbes from a carbonized organic material comprising:
a. inoculating a growth medium with a carbonized organic material containing spores of said fire-climax microbes;
b. incubating said growth medium to allow said spores to begin vegetative growth, thereby producing said fire-climax microbes.

7. The method of claim 6, wherein said carbonized organic material is a carbonized plant material.

8. The method of claim 7, wherein said carbonized plant material is charcoal.

9. The method of claim 8, wherein the charcoal is mesquite charcoal or almond charcoal.

10. The method of claim 6, wherein said carbonized organic material is carbonized oceanic organic waste.

11. The method of claim 6, wherein said carbonized organic material is produced by heating the organic material at about 600° C. for at least 10 minutes.

12. The method of claim 6, further comprising:
c. separating said fire-climax microbes from said growth medium.

13. The method of claim 6, further comprising
c. isolating individual strains from said fire-climax microbes produced in said growth medium.

14. A method of isolating fire-climax microbes from a soil sample comprising:
a. admixing said soil sample with water to obtain a liquid suspension;
b. boiling said liquid suspension;
c. inoculating a growth medium with an aliquot of the boiled suspension; and
d. maintaining the growth medium to permit vegetative growth of said fire-climax microbes within the medium.

15. The method of claim 14, further comprising:
e. separating said fire-climax microbes from said growth medium.

16. The method of claim 14, further comprising
e. isolating individual strains from said fire-climax microbes produced in said growth medium.

17. A composition comprising fire-climax microbes, wherein said fire-climax microbes are aerobic bacteria and produce spores that survive at a temperature of about 600° C.

18. The composition of claim 17, wherein said fire-climax microbes are isolated from a carbonized organic material, soil or a mixture thereof.

19. The composition of claim 18, wherein said fire-climax microbes are prepared according to any one of claims 6 or 14.

20. The composition of claim 17, wherein said fire-climax microbes are Bacillus sensu lato.

21. The composition of claim 20, wherein said fire-climax microbes comprise HAB7, AC9, or a combination thereof.

22. An isolated fire-climax microbe, wherein said fire-climax microbe is an aerobic bacteria and produces spores that survive at a temperature of about 600° C.

23. The isolated fire-climax microbe of claim 22, wherein said fire-climax microbe is isolated from charcoal, soil or a mixture thereof.

24. An isolated fire-climax microbe designated as HAB7 or AC9.

25. A method of identifying a fire-climax microbe capable of enhancing plant growth, comprising:
a. inoculating a growth medium with a carbonized organic material containing spores of fire-climax microbes;
b. incubating said growth medium to allow said spores to begin vegetative growth, thereby producing said fire-climax microbes;
c. isolating individual strains from said fire-climax microbes produced in step b;
d. determining the ability of said individual strains to enhance plant growth; and
e. identifying a fire-climax microbe strain that enhances plant growth.

26. The method of claim 25, wherein said carbonized organic material is a carbonized plant material.

27. The method of claim 26, wherein said carbonized plant material is charcoal.

28. The method of claim 27, wherein said charcoal is mesquite charcoal or almond charcoal.

29. The method of claim 25, wherein said carbonized organic material is carbonized oceanic waste.

30. The method of claim 25, wherein said carbonized organic material is produced by heating the organic material at about 600° C. for at least 10 minutes.

31. A method of identifying a fire-climax microbe capable of enhancing plant growth, comprising:
a. admixing a soil sample containing spores of fire-climax microbes with water to obtain a liquid suspension;
b. boiling said liquid suspension;
c. inoculating a growth medium with an aliquot of the boiled suspension;
d. incubating said growth medium to allow said spores to begin vegetative growth, thereby producing said fire-climax microbes;
e. isolating individual strains from said fire-climax microbes produced in step d;
f. determining the ability of said individual strains to enhance plant growth; and identifying a fire-climax microbe strain that enhances plant growth.

32. A method for stimulating plant growth and productivity, comprising growing cultivars of the plant in a plant cultivation medium supplemented with at least one fire-climax microbe, wherein said fire-climax microbe is an aerobic bacteria and produces spores that survive at a temperature of about 600° C.

33. The method of claim 32, wherein said plant cultivation medium is also supplemented with a carbonized plant material.

34. A method for increasing the nutrient or phytochemical content of a product of a plant, comprising growing cultivars of the plant in a plant cultivation medium supplemented with at least one fire-climax microbe, wherein said fire-climax microbe is an aerobic bacteria and produces spores that survive at a temperature of about 600° C.

35. The method of claim 34, wherein said plant cultivation medium is also supplemented with charcoal.

36. The method of claim 34, wherein the nutrient or phytochemical is a flavonoid or isoflavonoid.

37. The method of claim 34, wherein the nutrient or phytochemical is a vitamin.

38. The method of claim 34, wherein the vitamin is vitamin E (α-tocopherol or γ-tocopherol).

39. The method of claim 38, wherein the amount of α-tocopherol in said product of said plant is increased by at least 1.3-fold in comparison to a plant product grown in the absence of supplementation with said fire-climax microbe.

40. The method of claim 38, wherein the amount of γ-tocopherol is increased by at least 1.3-fold in comparison to a plant product grown in the absence of supplementation with said fire-climax microbe.

41. The method of claim 34, wherein the plant is an olive plant or an almond plant.

42. A nutritionally-enhanced plant product harvested from a plant grown according to the method of claim 34.

43. The nutritionally-enhanced plant product of claim 42, wherein the plant product is almond.

44. A method for improving the plant growth-promoting properties of a soil comprising applying to the soil, a composition containing at least one fire-climax microbe, wherein said fire-climax microbe is an aerobic bacteria and produces spores that survive at a temperature of about 600° C.

45. A method of relocating plant growth-stimulating fire-climax microbes present in a first soil to a second soil, comprising obtaining charcoal from plants grown in said first soil, and applying said charcoal to said second soil.

46. A method of relocating plant growth-stimulating fire-climax microbes present in a first soil to a second soil, comprising obtaining charcoal from plants grown in said first soil wherein said charcoal contains spores of said fire-climax microbes, inoculating a growth medium with said charcoal to initiate germination and vegetative growth of said spores to produce said fire-climax microbes, and applying said fire-climax microbes produced to said second soil.

47. A method of relocating plant growth-stimulating fire-climax microbes present in a first soil to a second soil, comprising obtaining charcoal from plants grown in said first soil wherein said charcoal contains spores of said fire-climax microbes, inoculating a growth medium with said charcoal to initiate germination and vegetative growth of said spores to produce said fire-climax microbes, identifying from the fire-climax microbes a strain that promotes plant growth characteristic of said first soil, and applying the identified strain to said second soil.

48. A method of relocating plant growth-stimulating fire-climax microbes present in a first soil to a second soil, comprising boiling a liquid suspension of said first soil, inoculating a growth medium with an aliquot of the boiled liquid suspension to initiate germination and vegetative growth of spores of said fire-climax microbes therein to produce said fire-climax microbes, and applying the fire-climax microbes produced to said second soil.

49. A method of relocating plant growth-stimulating fire-climax microbes present in a first soil to a second soil, comprising boiling a liquid suspension of said first soil, inoculating a growth medium with an aliquot of the boiled liquid suspension to initiate germination and vegetative growth of spores of said fire-climax microbes therein to produce said fire-climax microbes, identifying from the fire-climax microbes a strain that promotes plant growth characteristic of said first soil, and applying the identified strain to said second soil.

50. A method for enhancing growth and nutritional values of a plant, comprising growing cultivars of the plant in a plant cultivation medium supplemented with a carbonized plant material.

51. A method for improving the plant growth-promoting properties of a soil comprising applying carbonized plant material to the soil.

52. The method of claim 50 or 51, wherein said charcoal is selected for containing at least one fire-climax microbe, wherein said fire-climax microbe is an aerobic bacteria and produces spores that survive at a temperature of about 600° C.

53. A method of stimulating an animal's immune system comprising providing the plant product of claim 42 to said animal for consumption.

54. A method of enhancing the microbial flora and promoting health of an animal, comprising providing the plant product of claim 42 to said animal for consumption.

55. A method for enhancing solar energy conversion by plants, comprising growing cultivars of the plants in a plant cultivation medium supplemented with at least one fire-climax microbe isolated according to a method of any one of claims 6-16.

56. A method for reducing environmental pollution, comprising growing cultivars of plants in a plant cultivation medium supplemented with at least one fire-climax microorganism isolated according to a method of any one of claims 6-16.

57. A method of improving or restoring soil quality in a non-agricultural land, comprising applying to the soil, a composition containing at least one fire-climax microbe, wherein said fire-climax microbe is an aerobic bacteria and produces spores that survive at a temperature of about 600° C.

58. The method according to claim 57, wherein said composition is charcoal.

59. The method according to claim 57, wherein said non-agricultural land is an aesthetic wildland, an urban green belt or a golf course.


SEQUENCE LISTING (1 sequence)

SEQ ID NO: 1 - DNA, 1531 bp, Brevibacillus centrosporus
tggagagtttgatcctggctcaggacgaacgctggcggcgtgcctaatacatgcaagtcgagcgagtctcttcggaggctagcggcggacgggtgagtaacacgtaggcaacctgcccgtaagactgggataacatagggaaacttatgctaataccagatagagtctactctcgcatgagggaagacggaaaggtggcgcaagctatcacttacggatgggcctgcggcgcattagctagttggtggggtaatggcctaccaaggcgacgatgcgtagccgacctgagagggtgaccggccacactgggactgagacacggcccagactcctacgggaggcagcagtagggaattttccacaatggacgaaagtctgatggagcaacgccgcgtgaacgatgaaggtcttcggattgtaaagttctgttgttagggacgaacaagtaccgttcgaayagggcggtaccttgacggtacctgacgagaaagccacggctaactacgtgccagcagccgcggtaatacgtaggtggcaagcgttgtccggatttattgggcgtaaagcgcgcgcaggcggctatgtaagtctggtgttaaagcccggagctcaactccggttcgcatcggaaactgtgtagcttgagtgcagaagaggaaagcggtattccacgtgtagcggtgaaatgcgtagagatgtggaggaacaccagtggcgaaggcggctttctggtctgtaactgacgctgaggcgcgaaagcgtggggagcaaacaggattagataccctggtagtccacgccgtaaacgatgagtgctaggtgttgggggtttcaataccctcagtgccgcagctaacgcaataagcactccgcctggggagtacgctcgcaagagtgaaactcaaaggaattgacgggggcccgcacaagcggtggagcatgtggtttaattcgaagcaacgcgaagaaccttaccaggtcttgacatcccgctgaccgctctggagacagagcttcccttcggggcagcggtgacaggtggtgcatggttgtcgtcagctcgtgtcgtgagatgttgggttaagtcccgcaacgagcgcaacccttatttctagttgccagcattcagttgggcactctagagagactgccgtcgacaagacggaggaaggcggggatgacgtcaaatcatcatgccccttatgacctgggctacacacgtgctacaatggttggtacaacgggatgctacctcgcgagaggacgccaatctcttaaaaccaatctcagttcggattgtaggctgcaactcgcctacatgaagtcggaatcgctagtaatcgcggatcagcatgccgcggtgaatacgttcccgggccttgtacacaccgcccgtcacaccacgggagtttgcaacacccgaagtcggtgaggtaaccgcaaggagccagccgccgaaggtggggtagatgactggggtgaagtcgtaacaaggtatccgtaccggaaggtgcggctggatcacctcctt